(19) United States
(12) Patent Application Publication
(10) Pub. No.: US 2024/0153088 A1
(43) Pub. Date: May 9, 2024

(54) MEDICAL IMAGE ANALYSIS APPARATUS, MEDICAL IMAGE ANALYSIS METHOD, AND MEDICAL IMAGE ANALYSIS SYSTEM

(71) Applicant: SONY GROUP CORPORATION, TOKYO (JP)

(72) Inventors: HIROKI DANJO, TOKYO (JP); KAZUKI AISAKA, TOKYO (JP); TOYA TERAMOTO, TOKYO (JP); KENJI YAMANE, TOKYO (JP)

(21) Appl. No.: 18/550,298

(22) PCT Filed: Feb. 17, 2022
(86) PCT No.: PCT/JP2022/006290

§ 371 Date: Sep. 13, 2023

(30) Foreign Application Priority Data
Mar. 24, 2021 (JP) 2021-049872

(51) Int. Cl. G06T 7/00

(52) U.S. Cl. CPC G06T 7/0014

(57) ABSTRACT

In parallel with an operation of browsing a pathological-tissue image, retrieval of similar cases from past cases using image information about the pathological-tissue image is automatically performed. An analysis apparatus of the present disclosure includes a first setting unit configured to set sample regions in an analysis target region of an image obtained by imaging of a biologically-originated sample, on the basis of an algorithm; a processing unit configured to select at least one reference image from a plurality of reference images associated with a plurality of cases, on the basis of images of the sample regions; and an output unit configured to output the selected reference image.

### TECHNICAL FIELD

The present disclosure relates to a medical image analysis apparatus, a medical image analysis method, and a medical image analysis system.

### BACKGROUND ART

When performing diagnosis on a pathological-tissue image of each case in a clinical site, a surgeon such as a pathologist browses the pathological-tissue image while referring to information regarding a place of interest in the browsed image (for example, images of similar cases in the past and information thereabout) at the same time. However, there are discrepancies in consciousness and time between browsing of the pathological-tissue image and referring to relevant information. Repetition of such work increases a burden on the pathologist and is not an efficient workflow. Patent Document 1 discloses an apparatus that uses an image of a pathological tissue as an input, retrieves a similar image from an image database using construction information about cell nuclei in the input image, and outputs the retrieved image together with opinion data.

### CITATION LIST

### Patent Document

Patent Document 1: Japanese Patent Application Laid-Open No. 2009-9290

### SUMMARY OF THE INVENTION

### Problems to be Solved by the Invention

However, in Patent Document 1, no detailed reference to an image to be input is made, and a pathologist cannot always refer to information regarding a place of interest.

An object of the present disclosure is to improve efficiency of work of a surgeon who performs diagnosis using an image.

### Solutions to Problems

A medical image analysis according to the present disclosure includes: a first setting unit configured to set sample regions in an analysis target region of an image obtained by imaging of a biologically-originated sample, on the basis of an algorithm; a processing unit configured to select at least one reference image from a plurality of reference images associated with a plurality of cases, on the basis of images of the sample regions; and an output unit configured to output the selected reference image.

A medical image analysis system according to the present disclosure includes: an imaging device configured to image a biologically-originated sample; a first setting unit configured to set sample regions in an analysis target region of an image acquired by the imaging device, on the basis of an algorithm; a processing unit configured to select at least one reference image from a plurality of reference images associated with a plurality of cases, on the basis of images of the sample regions; and an output unit configured to output the selected reference image.

A medical image analysis method according to the present disclosure includes: setting sample regions in an analysis target region of an image obtained by imaging of a biologically-originated sample, on the basis of an algorithm: selecting at least one reference image from a plurality of reference images associated with a plurality of cases, on the basis of images of the sample regions; and outputting the selected reference image.

### BRIEF DESCRIPTION OF DRAWINGS

FIG. 1 is a block diagram of a medical image analysis system including a medical image analysis apparatus according to an embodiment of the present disclosure.

FIG. 2 is a view illustrating an example in which an analysis target region is set.

FIG. 3 is a view illustrating an example in which a plurality of sub-regions (sample regions) are set in an analysis target region.

FIG. 4 is a view illustrating an example in which retrieval results of sub-regions are arranged in a descending order of a similarity.

FIG. 5 is a view illustrating an example of a case information screen displayed by a case information display unit.

FIG. 6 is a view illustrating an example in which a user who is browsing the screen of FIG. 5 changes case information to be displayed.

FIG. 7 is a view schematically illustrating an example in which clinical information or the like, or statistical information or the like, corresponding to a pathological-tissue image being browsed, is displayed.

FIG. 8 is a view illustrating an example in which a small-section image in a past case and a pathological-tissue image are displayed side by side.

FIG. 9 is a view illustrating various display examples of a sub-region.

FIG. 10 is a flowchart schematically illustrating an example of overall operations of the analysis apparatus of the present disclosure.

FIG. 11 is a flowchart illustrating an example of detailed operations of a pathological-tissue image display unit and an analysis-target-region setting unit.

FIG. 12 is a flowchart illustrating an example of detailed operations of a sub-region setting unit.

FIG. 13 is a flowchart illustrating an example of detailed operations of a similar-case retrieval unit.

FIG. 14 is a flowchart illustrating an example of a display operation performed in a case where a user selects a small-section image of a similar case on the case information screen.

FIG. 15 is a flowchart illustrating an example of operations performed in a case where the similar-case retrieval unit analyzes information about a similar case and displays a result of analysis with a case information display unit.

FIG. 16 is a view illustrating an example of a configuration of an analysis system according to the present disclosure.

FIG. 17 is a view illustrating an example of an imaging method.

### MODE FOR CARRYING OUT THE INVENTION

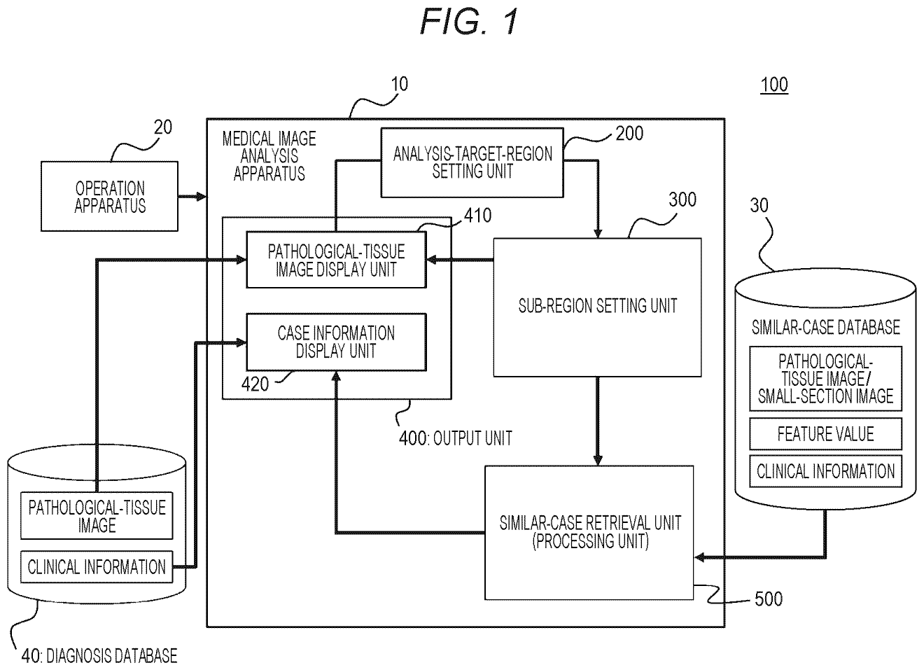

FIG. 1 is a block diagram of a medical image analysis system 100 including a medical image analysis apparatus 10 according to an embodiment of the present disclosure.

The medical image analysis system 100 includes the medical image analysis apparatus 10, an operation apparatus 20, a similar-case database 30, and a diagnosis database 40. The medical image analysis apparatus 10 includes an analysis-target-region setting unit 200 (second setting unit), a sub-region setting unit 300 (first setting unit), an output unit 400, and a similar-case retrieval unit 500 (processing unit). The output unit 400 includes a pathological-tissue image display unit 410 and a case information display unit 420. The medical image analysis apparatus 10 executes a medical image analysis application (hereinafter also referred to as the present application) used by a user of the medical image analysis apparatus 10. The user of the medical image analysis apparatus 10 is a surgeon such as a pathologist, but the user is not limited to a surgeon, and may be, for example, a person following a surgeon. The output unit 400 generates screen data of the present application and causes a display (for example, a liquid crystal display device, an organic EL display device, or the like) to display the image data. In the present embodiment, the display is included in the output unit 400, but may be connected to the medical image analysis apparatus 10 from the outside of the medical image analysis apparatus 10 by wires or wirelessly. In such a case, the output unit 400 is only required to transmit the image data to the display by wires or wirelessly.

The medical image analysis apparatus 10 is connected to the similar-case database 30 (similar-case DB 30) and the diagnosis database 40 (diagnosis DB 40) by wires or wirelessly. The medical image analysis apparatus 10 can read or acquire information from the diagnosis DB 40 and the similar-case DB 30. The medical image analysis apparatus 10 can write or transmit information to the diagnosis DB 40 and the similar-case DB 30. The diagnosis DB 40 and the similar-case DB 30 may be formed integrally with each other.

The medical image analysis apparatus 10 may be connected to the diagnosis DB 40 and the similar-case DB 30 via a communication network such as the Internet or an intranet, or via a cable such as a USB cable. Alternatively, the diagnosis DB 40 and the similar-case DB 30 may be included in the medical image analysis apparatus 10, as a part of the medical image analysis apparatus 10.

The medical image analysis apparatus 10 is connected to the operation apparatus 20 by wires or wirelessly. The operation apparatus 20 is operated by the user of the medical image analysis apparatus 10. The user inputs various instructions as input information to the medical image analysis apparatus 10 using the operation apparatus 20. The operation apparatus 20 may be any device such as a keyboard, a mouse, a touch panel, a voice input device, or a gesture input device.

The diagnosis DB 40 is a database in which diagnosis information is stored. The diagnostic information includes, for example, information regarding a case of a subject, such as a pathological-tissue image and clinical information of the subject. The diagnosis information may include other information. The diagnosis DB 40 includes, for example, a memory device, a hard disk, an optical recording medium or a magnetic recording medium, and the like. Here, a pathological-tissue image is an image obtained by imaging of a biologically-originated sample (hereinafter referred to as a biologically-originated sample S). Below, the biologically-originated sample S will be described.

(Biologically-Originated Sample)

The biologically-originated sample S may be a sample containing a biological component. The biological component may be a tissue or a cell of a living body, a liquid component of a living body (blood, urine, and the like), a culture, or a living cell (such as a cardiomyocyte, a nerve cell, and a fertilized egg).

The biologically-originated sample S may be a solid, and may be a specimen immobilized by an immobilizing reagent such as paraffin, or a solid formed by freezing. The biologically-originated sample S can be a section of the solid. Specific examples of the biologically-originated sample S include a section of a biopsy sample.

The biologically-originated sample S may be one having been subjected to treatment such as staining or labeling. The treatment may be staining for showing a form of a biological component or showing a substance contained in a biological component (such as a surface antigen), and examples thereof can include hematoxylin-eosin (HE) staining and immunohistochemistry staining. The biologically-originated sample S may be one having been subjected to the treatment with one or two or more reagents, and the reagent can be a fluorescent dye, a coloring reagent, a fluorescent protein, or a fluorescently-labeled antibody.

The specimen may be prepared from a specimen or a tissue sample collected from a human body for the purpose of pathological diagnosis, clinical examination, or the like. Furthermore, the specimen is not limited to a human body, and may be originated from an animal, a plant, or another material. The specimen has different properties depending on a type of tissue to be used (for example, an organ, a cell, or the like), a type of a target disease, an attribute of a subject (for example, an age, a sex, a blood type, a race, or the like), a lifestyle of a subject (for example, a dietary habit, an exercise habit, a smoking habit, or the like), or the like. The specimen may be managed by being affixed with identification information (bar-code information, QR-code (trademark) information, or the like) by which each specimen can be identified.

The diagnosis DB 40 can provide diagnosis information to the medical image analysis apparatus 10. Furthermore, in the diagnosis DB 40, a part or all of analysis result data of the medical image analysis apparatus 10 may be stored as new information regarding a case of a subject.

The similar-case DB 30 is a database in which information about various past cases of various subjects are stored. The information about various cases includes, for example, pathological-tissue images and clinical information regarding a plurality of cases. Further, the information about various cases includes a feature value calculated on the basis of a small-section image that is a part of a pathological-tissue image. A small-section image (or pathological-tissue image) corresponds to an example of a reference image associated with a plurality of cases. In addition, in a case where the operations of the medical image analysis apparatus 10 are performed by a computer (for example, a processor such as a central processing unit (CPU)), the similar-case DB 30 may include operation data such as a computer program executed by the computer, and parameters.

The similar-case DB 30 can provide information regarding various past cases to the medical image analysis apparatus 10. Furthermore, in the similar-case DB 30, all or a part of analysis result data of the medical image analysis apparatus 10 may be stored as new information regarding a case, and may be used as information regarding a past case the next time and later.

(Pathological-Tissue Image Display Unit 410)

The pathological-tissue image display unit 410 displays a part or all of a pathological-tissue image that is specified by the user of the present application with the use of the operation apparatus 20, on a part (first screen portion) of the screen of the present application. A screen displaying a part or all of a pathological-tissue image (a display region of a pathological-tissue image) is referred to as a pathological-tissue browsing screen. The medical image analysis apparatus 10 reads the pathological-tissue image specified by the user from the diagnosis DB 40 and displays the image on the pathological-tissue browsing screen in a window of the present application. In a case where the pathological-tissue image has a size larger than the pathological-tissue browsing screen and only a part of the pathological-tissue image is displayed in the pathological-tissue browsing screen, the pathological-tissue image can be moved by a mouse operation by the user or the like so that the image displayed in the pathological-tissue browsing screen can be changed. The user can check the state of the pathological tissue by browsing the image displayed in the pathological-tissue browsing screen. Furthermore, the user may be capable of changing the magnification of the image while browsing the image. In this case, it is only required that the user reads an image at a magnification specified by the user from the diagnosis DB 40, and re-displays the image on the pathological-tissue browsing screen.

(Analysis-Target-Region Setting Unit 200)

The analysis-target-region setting unit 200 sets (or registers) an analysis target region in a part or all of an image displayed in the pathological-tissue browsing screen in a pathological-tissue image. The analysis-target-region setting unit 200 is an example of a second setting unit configured to set an analysis target region in a pathological-tissue image. The analysis-target-region setting unit 200 may set a region of an image corresponding to a predetermined range (all or a part) in the pathological-tissue browsing screen, as an analysis target region. Alternatively, the analysis-target-region setting unit 200 may set a region of interest of the user in an image displayed on the pathological-tissue browsing screen, as an analysis target region. A region of interest may be determined on the basis of instruction information from the user provided from the operation apparatus 20. For example, a place specified by the user in an image may be set as a region of interest. For example, in a case where the user encloses a region of interest with a rectangle or the like through a mouse operation or the like, the enclosed region may be set as a region of interest. Alternatively, a region that has predetermined vertical and horizontal widths around a point indicated by the user via a click or the like in an image, as center coordinates thereof, may be set as a region of interest. Further alternatively, a range that is algorithmically determined around a point indicated by the user, as center coordinates thereof, may be set as a region of interest. For example, in a case where the number of cells included in a part of a region enclosed in a certain range from the center coordinates thereof is the maximum value, the part of the region is set as a region of interest. However, the algorithm is not limited to a specific algorithm.

The analysis-target-region setting unit 200 may automatically set an analysis target region on the basis of a detection algorithm. For example, in a case where there is no movement (change) of an image for a certain period of time or more in the pathological-tissue browsing screen, or in a predetermined region (region defined in advance) of the pathological-tissue browsing screen, a region of an image corresponding to the predetermined region (region of an image included in the predetermined region) may be set as an analysis target region. An example of the predetermined region may be a region in a certain range from the center of the pathological-issue browsing screen (a display region of a pathological-tissue image), or may be another region. Despite movement of the image in the predetermined region, on condition that the amount of the movement is equal to or less than a threshold pixel, a region of the image corresponding to the predetermined region after a lapse of a certain period of time may be set as an analysis target region. In addition, it is also possible to use a method in which a line-of-sight detection sensor is provided in the medical image analysis apparatus 10, and a region of an image portion to which the user's line of sight is directed for a certain period of time or more is detected as an analysis target region. As described above, in a case where a method in which an analysis target region is set without the user's operation is used, there is produced an advantage of allowing the user to concentrate on browsing a pathological-tissue image.

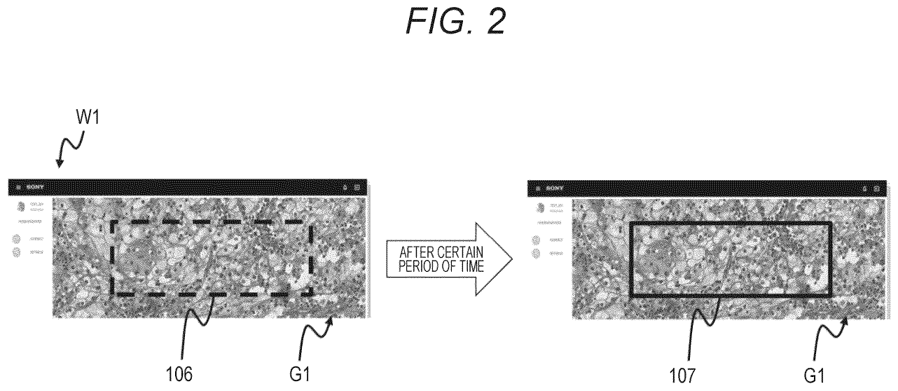

FIG. 2 illustrates a specific example in which an analysis target region is set. In FIG. 2, all or a part of a pathological-tissue image (slide image) selected by the user is displayed on a pathological-tissue browsing screen G1 in a window W1. The position of the image in the pathological-tissue browsing screen G1 may be changeable by the user's operation. In the image, a region 106 is indicated by a broken line. The region 106 is a region that is a candidate for an analysis target region. One example of the region 106 is a predetermined region including the center of the pathological-tissue browsing screen G1. Alternatively, the region 106 may be a region marked by the user via dragging or the like (a region specified by the user as a region of interest). In a case where there is no change (movement) in the image in the region 106 for a certain period of time, in other words, in a case where the same image is kept present in the region 106, the analysis-target-region setting unit 200 sets the region 106 in the displayed image as an analysis target region 107. An analysis target region may be set by a method other than the method illustrated in FIG. 2.

(Sub-Region Setting Unit 300)

A sub-region setting unit 300 sets one or more sub-regions (sample regions) in an analysis target region on the basis of an algorithm for setting a sample region. The sub-region setting unit 300 includes a first setting unit configured to set one or more sub-regions (sample regions) in an analysis target region. Specifically, the first setting unit sets one or more sample regions in an analysis target region of an image obtained by imaging of a biologically-originated sample, on the basis of the algorithm. A sub-region is a region having a size smaller than that of an analysis target region. A sub-region is a region serving as a unit for extracting a feature value of an image. The shape of a sub-region is not limited to any particular shape, and may be a rectangle, a circle, an ellipse, a shape defined in advance by the user, or the like.

In a specific example in which a sub-region is set, reference coordinates are set in random positions or at regular intervals in an analysis target region, and a rectangular region that has predetermined vertical and horizontal widths around a center at the reference coordinates is set as a sub-region. The number of coordinated points of the reference coordinates is not limited to any particular number. As described, sub-regions may be set in random positions or may be set at regular intervals. The size of a sub-region may be determined in advance or may be determined in accordance with a magnification of an image. Furthermore, the size of a sub-region may be specifiable by the user.

A process of selecting a sub-region expected to be effective for retrieving a past similar case may be performed on sub-regions having been set. For example, the density of cell nuclei is calculated for each sub-region. Then, a sub-region having a density equal to or greater than a threshold value, or less than the threshold value, may be employed. The sub-region that has not been employed is not used in a later process. Likewise, the size of a cell nucleus is calculated for each sub-region. Then, a sub-region having a statistical value (maximum value, average value, minimum value, median value, or the like) of the size that is equal to or less than a threshold value, or equal to or greater than the threshold value, may be employed. The sub-region that has not been employed is not used in a later process.

A plurality of sub-regions is clustered on the basis of a feature-value distribution for each sub-region, to form a plurality of clusters. Then, one or more sub-regions may be selected from each cluster as a sub-region representing the cluster. The number of sub-regions selected from each cluster may be the same. For the feature-value distribution, for example, an image feature value such as a luminance distribution, an RGB distribution, a brightness distribution, or a saturation distribution may be used. In addition, a cytological feature value such as a cell-number distribution, a cell-density distribution, or a heteromorphic-degree distribution may be used.

The setting of a sub-region is not limited to being performed on an image at a magnification at which the user is browsing. For example, the setting of a sub-region may be performed on an image at a magnification effective for a case that the user desires to retrieve, different from a magnification at which the user is browsing. One magnification or a plurality of magnifications may be used. For example, in a case where a magnification of an image being browsed by the user is a low magnification not suitable for a case that the user desires to retrieve, an image at a high magnification is read, and a sub-region (a region at coordinates corresponding to coordinates of a sub-region set in the image being browsed) is set in the read image. In a case where a magnification at which the user is browsing is not effective for a case that the user desires to retrieve, there is no need of acquiring an image from the sub-region set in the image being browsed by the user.

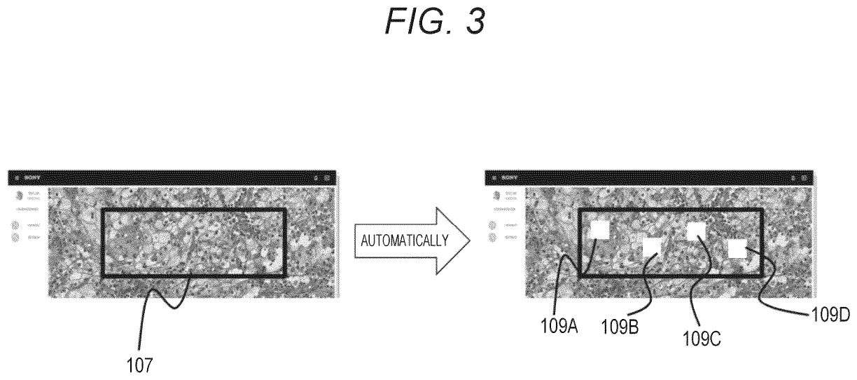

FIG. 3 illustrates an example in which a plurality of sub-regions (sample regions) is set in the analysis target region 107. In the example of the drawing, four sub-regions 109A to 109D are set. The sub-regions illustrated in the drawing are square or rectangular, but may have other shapes such as a circular shape. The sub-regions 109A to 109D are automatically set on the basis of the algorithm as described above.

The sub-region setting unit 300 acquires (cuts out) an image of each sub-region set in the analysis target region 107, and provides the acquired image, that is, a small-section image (image of a sample region) or the like, to the similar-case retrieval unit 500. The sub-region setting unit 300 includes an acquisition unit configured to acquire an image from a sub-region (sample region). The acquisition unit may be included in the similar-case retrieval unit 500 (processing unit) described later. As described above, an image of a sub-region (small-section image) may be acquired from an image at a magnification determined in accordance with a case that is desired to be retrieved. For example, regarding a sub-region set in an image being browsed at a 20× magnification, a region corresponding to the sub-region is specified in an image at a 40× magnification (the specified region is defined as a sub-region), and the image of the specified sub-region may be acquired as a small-section image. The sub-region setting unit 300 may determine one magnification or a plurality of magnifications corresponding to a case that the user desires to retrieve, on the basis of data that defines a correspondence between a case and at least one magnification. The magnification corresponding to a case to be retrieved is not necessarily required to be one, but there may be a plurality of magnifications. In this case, a small-section image can be acquired from an image for each magnification. Thus, by distinguishing a magnification at which the user is browsing and a magnification used for retrieving a similar case from each other, it is possible to improve the work efficiency of the user, and at the same time, improve the accuracy of analysis.

(Similar-Case Retrieval Unit 500)

The similar-case retrieval unit 500 acquires an image of each sub-region from the sub-region setting unit 300, and acquires a small-section image (a part of a pathological-tissue image of a past case) similar to the acquired image of each sub-region from the similar-case DB 30 (similar-case retrieval). The similar-case retrieval unit 500 corresponds to an example of a processing unit configured to select at least one reference image from a plurality of reference images associated with a plurality of cases on the basis of an image of a sample region. A small-section image stored in the similar-case DB 30 is an image obtained by cutting-out of a part of a pathological-tissue image of a past case stored in the similar-case DB 30. Each small-section image in the similar-case DB 30 has been cut out from an original pathological-tissue image, and clinical information is also attached to the pathological-tissue image. That is, the similar-case retrieval is to retrieve a small-section image of a past case that is similar to an image of a sub-region eventually. The similar-case retrieval unit 500 acquires information about a past case similar to an image of each sub-region (small-section image, clinical information, and the like), as a result of retrieval of the sub-region, and generates an analysis result for an analysis target region on the basis of the result of retrieval of the sub-region. Below, the similar-case retrieval unit 500 will be described in more detail.

The similar-case retrieval unit 500 calculates a similarity between the acquired image of each sub-region and a small-section image of a past case in the similar-case DB 30. In order to calculate a similarity between an image of each sub-region and a small-section image of a past case, the similar-case retrieval unit 500 compares a feature value of an image of each sub-region with a feature value of a small-section image of a past case. One example of a feature value is a vector indicating a feature of an image.

Examples of a feature value that can be interpreted by humans include the number of cells, a sum of distances between cells, and a result obtained by integration of those using a vector including a cell density, a machine learning method, or the like. In this case, a feature value may be calculated using a computer program for calculating the number of cells, a sum of distances between cells, and a cell density.

Meanwhile, an example of a feature value that cannot be (generally) interpreted by humans is a vector in which numerical values are arranged, for example, a high-dimensional vector such as a 2048 dimensional vector. In this case, a Deep learning model that can perform classification for each label using an image as an input can be used. In addition to a Deep learning model, a model based on a general machine learning method may be used.

One of methods of comparing feature values to calculate a similarity is to calculate a distance (difference) between vectors and set a value corresponding to the distance as a similarity. Note that it is supposed that the feature values to be compared are calculated by the same algorithm. For example, it is supposed that the shorter the distance, the more similar (the higher the similarity), and the longer the distance, the less similar (the lower the similarity). Specific examples of a similarity include Euclidean distance, a cosine similarity, and the like, but other measures may be used. Depending on the definition of a similarity, the larger the value of a similarity, the higher the similarity in some cases, and the smaller the value of a similarity, the higher the similarity in other cases. Feature values may be compared between images at the same magnification. In the similar-case DB 30, small-section images and pathological-tissue images of past cases at a plurality of magnifications may be stored. This enables higher retrieval accuracy.

Feature values are compared with each other in order to calculate a similarity, but a similarity may be calculated by direct comparison of an image of a sub-region with a small-section image of a past case without calculation of a feature value. For example, a learned model (machine learning model) may be used to calculate a similarity between an image of a sub-region and a small-section image of a past case. As a machine learning model, for example, a neural network, a multiple regression model, and a regression model such as a decision tree can be used. For example, an image of a sub-region and a small-section image of a past case are input to a neural network, and a similarity between the images is output. A machine learning model may be prepared for each case to be retrieved in one example. Alternatively, a machine learning model may be prepared for each magnification of an image. This enables higher retrieval accuracy.

The similar-case retrieval unit 500 generates an analysis result for an analysis target region on the basis of a similarity to each small-section image of a past case, calculated for an image of each sub-region. Among the small-section images of past cases for which a similarity is calculated, a case of a small-section image having a similarity equal to or higher than a threshold, or a certain number of cases of small-section images each having a higher similarity, will be referred to as a similar case or similar cases. A small-section image, clinical information, and the like of a similar case will be referred to as a retrieval result for a sub-region. Below, a method of generating an analysis result of an analysis target region will be described in detail.

(First method) For example, for each sub-region, a series of retrieval results in which retrieval results of a sub-region (small-section images of past cases, clinical information, and the like) are arranged in a descending order of a similarity is defined as an analysis result for an analysis target region. By outputting the retrieval results for each sub-region, it is possible to allow the user to easily understand each sub-region in a pathological-tissue image being browsed and a past case similar to each sub-region in association with each other. Furthermore, by sequentially outputting the retrieval results for each sub-region from the most similar one, it is possible to allow the user to preferentially refer to a small-section image that is most likely to be a case that the user desires to retrieve.

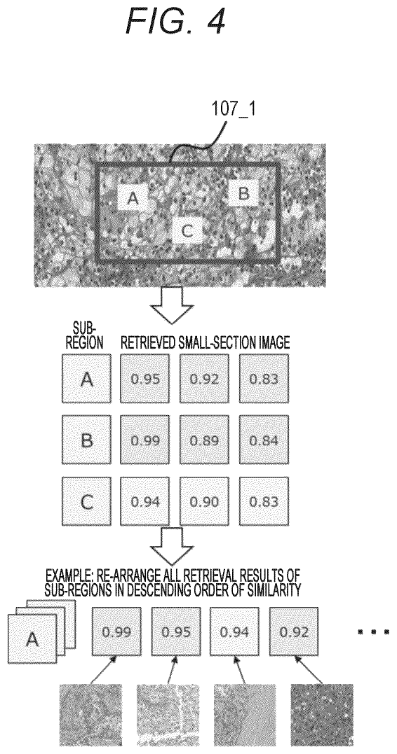

(Second method) A series of retrieval results in which retrieval results of all sub-regions are arranged in a descending order of a similarity calculated for all the sub-regions is defined as an analysis result for an analysis target region. In the above-described first method, the retrieval results are arranged for each sub-region. On the other hand, in the second method, the retrieval results of all the sub-regions are arranged according to the similarities thereof. The retrieval results of all the sub-regions are integrated in this manner, to generate an analysis result for an analysis target region. As a result, the average feature of a pathological tissue included in an analysis target region can be output. Furthermore, the user can preferentially refer to a retrieval result that is most likely to be a case that the user desires to retrieve. A specific example of the second method will be described with reference to FIG. 4.

FIG. 4 is a view illustrating an example in which retrieval results of all sub-regions are arranged in a descending order of a similarity calculated for all the sub-regions. Sub-regions A, B, and C are set in an analysis target region 107_1. For the sub-region A, three small-section images having the highest similarities (similarities are 0.95, 0.92, and 0.83, respectively) among small-section images of past cases are selected. For the sub-region B, three small-section images having the highest similarities (similarities are 0.99, 0.89, and 0.84, respectively) among small-section images of past cases are selected. For the sub-region C, three small-section images having the highest similarities (similarities are 0.94, 0.90, and 0.83, respectively) among small-section images of past cases are selected. A descending order of a similarity for all the sub-regions A to C is 0.99, 0.95, 0.94, 0.92 . . . . At the bottom of the drawing, small-section images of past cases for which the above-mentioned similarities have been calculated are illustrated.

(Third method) For each sub-region, a small-section image having a similarity equal to or higher than a threshold, or a certain number of small-section images each having a higher similarity, are selected. Then, the selected small-section image is evaluated. For example, the cell-density distribution of a set of all sub-regions in an analysis target region is calculated and compared with the cell-density distribution of the above-described selected small-section image (for example, a distance between the distributions is calculated). On the basis of the distance between the distributions, a small-section image similar to the distributions of all the sub-regions (for example, a small-section image in which the distance between the distributions is less than a threshold) is selected. The selected small-section image and clinical information or the like for the selected small-section image are defined as a re-retrieval result, to generate an analysis result of the analysis target region.

(Case Information Display Unit 420)

The case information display unit 420 displays case information based on an analysis result for an analysis target region on a case information screen in the window W1 of the present application. The case information screen corresponds to a second screen portion of the screen of the present application.

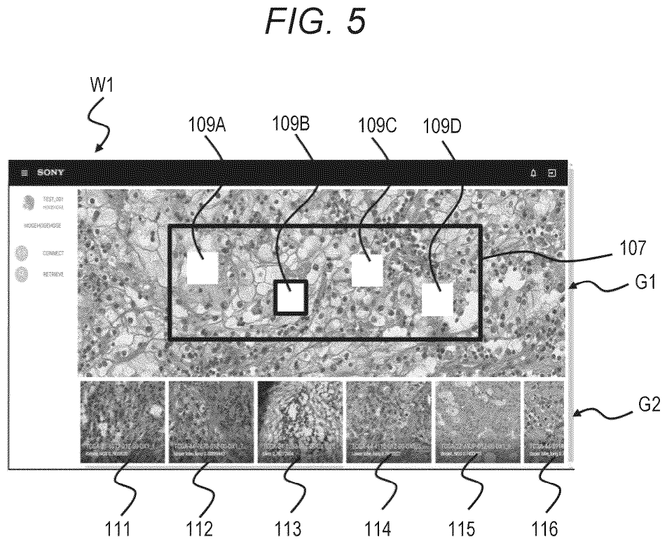

FIG. 5 illustrates an example of a case information screen G2 displayed by the case information display unit 420 on the lower side in the pathological-tissue browsing screen G1 (first screen portion) in the window W1 of the present application. On the pathological-tissue browsing screen G1 (first screen portion), a pathological-tissue image, the analysis target region 107, and the sub-regions 109A to 109B are displayed. One of the sub-regions 109A to 109D can be selected on the basis of instruction information from the user, and in the example illustrated in the drawing, the sub-region 109B is selected by the user. In the case information screen G2 (second screen portion) on the lower side in the window W1, case information based on an analysis result for the analysis target region is displayed. In the example illustrated in the drawing, as the case information, small-section images 111 to 116 (111,112,113,114,115,116) of past cases are sequentially arranged from the left in an order from a small-section image having the highest similarity to the sub-region 109B selected by the user. In this example, the user selects the sub-region 109B, but the user can select another sub-region. In such a case, in accordance with another sub-region having been selected, the small-section images are sequentially arranged and displayed from the left on the case information screen G2 in an order from a small-section image having the highest similarity. In the example illustrated FIG. 5, a small-section image is displayed as case information, but clinical information regarding the small-section image may be further displayed. Alternatively, in a case where the user gives instruction information for referring to the attribute of a small-section image, clinical information may be displayed as a pop-up menu or the like.

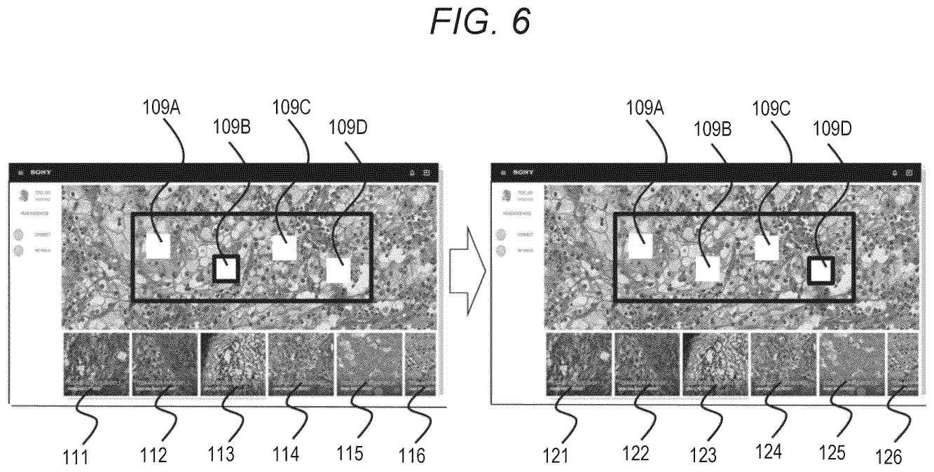

FIG. 6 illustrates an example in which, in a case where a sub-region selected by the user who is browsing the window W1 in FIG. 5 is changed from the sub-region B to the sub-region D, case information to be displayed is changed in response to the change of a sub-region. Small-section images 121 to 126 (121,122,123,124,125,126) of past cases are displayed in an order from a small-section image having the highest similarity to the sub-region D. By such a change of case information to be displayed, the user can compare retrieval results for various sub-regions, and can deepen understanding as compared to a case where a retrieval result for a single sub-region is referred to. Furthermore, in a case where a specific sub-region is not selected, a retrieval result for an analysis target region generated by any of the above-described first to third methods or the like may be displayed.

Note that the user may perform an operation of deleting a frame of the analysis target region 107 and the display of the sub-regions 109A to 109D from the pathological-tissue browsing screen G1 to delete the frame and the display.

Furthermore, information regarding a pathological-tissue image being browsed (for example, clinical information) may be displayed in the window W1. The clinical information may include an image distribution of the pathological-tissue image, cell information such as the number of cells, and the like. The output unit 400 may statistically process those pieces of information in response to instruction information from the user and display the result data of the statistical process. The user may input instruction information for analysis by clicking an analysis button separately provided in the window W1. The statistical information may be displayed in graph form (line graph, circle graph, or the like) or in text form. Data such as statistical information may be displayed on an analysis screen in the window W1 or may be displayed on a pop-up screen. Alternatively, the data may be displayed by another method in which the data is superposed on a pathological-tissue image, or the like.

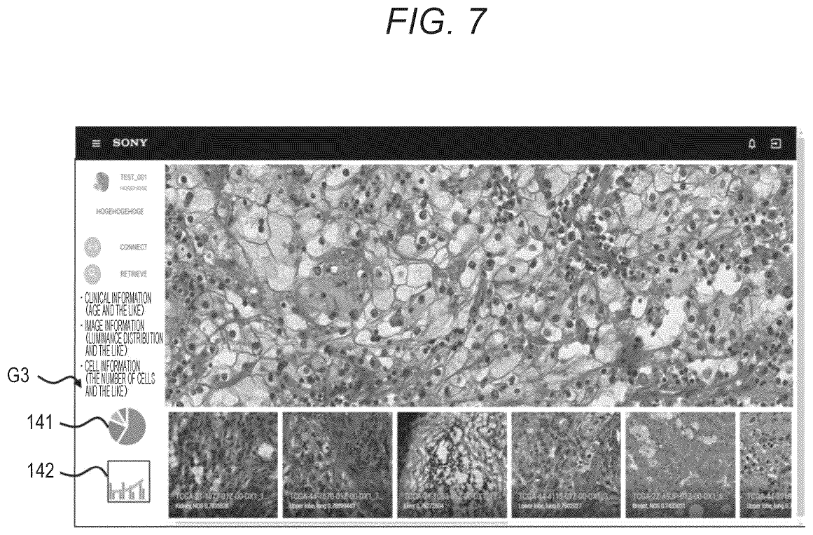

FIG. 7 schematically illustrates an example in which information regarding a pathological-tissue image being browsed is displayed on an analysis screen (analysis window) G3 on the left side in the window W1. In this example, the age and the like of a subject, a luminance distribution of the pathological-tissue image, the number of cells, and the like are displayed. For example, a result of statistical analysis of an image feature or a cytological feature of the pathological-tissue image and information obtained from other images may be displayed in graph form or the like (see a circle graph 141, a bar graph 142, and the like at the lower left in the drawing). As a result, the user can easily understand the tendency or the like regarding clinical information. The analysis screen G3 may be another screen such as a pop screen different from the window W1.

Furthermore, clinical information about a similar case or data obtained by a statistical process of the clinical information may be displayed in a randomly-selected region (for example, the analysis screen G3 described above) in the window W1. For example, the output unit 400 may display all or a part of the clinical information corresponding to a case of each retrieval result in the window W1 or another screen. A result of statistically processing the clinical information may be displayed in text form, graph form, or the like (see the circle graph 141, the bar graph 142, and the like at the lower left in FIG. 7). Furthermore, it is possible to encourage the user to more deeply understand the result retrieved as the similar case. In addition, the feature value of an image regarding a sub-region and cell information such as the number of cells, or data obtained by a statistical process of the feature value and the cell information, or the like may be displayed in text form, graph form, or the like. As a result, the user can easily understand what kind of feature the selected sub-region has in the sub-region setting unit 300.

In a case where the user selects any of the small-section images displayed on the case information screen G2 on the lower side in the window W1, the selected small-section image and the pathological-tissue image may be arranged and displayed side by side. At that time, the small-section image may be enlarged. Furthermore, a pathological-tissue image including a sub-region corresponding to the selected small-section image may be displayed such that the sub-region is located at or near the center in the display region. Furthermore, for the pathological-tissue image to be displayed, the display position of the pathological-tissue image may be adjusted such that a sub-region related to the selected small-section image (sub-region for which a similarity of the small-section image has been calculated) is located at or near the center of the display region of the pathological-tissue image. This facilitates comparison between the sub-region and the selected small-section image. Furthermore, by displaying the pathological-tissue image including the sub-region corresponding to the selected small-section image next to the selected small-section image and browsing the two types of pathological-tissue images arranged side by side, it is also possible to observe a region outside the sub-region related to the selected small-section image.

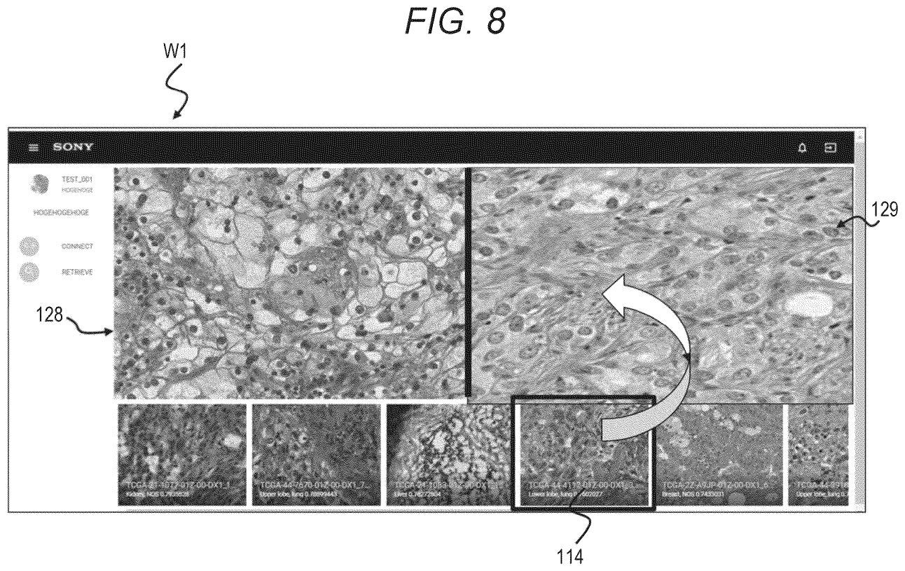

FIG. 8 illustrates an example in which, in a case where the user selects the small-section image 114 by clicking or the like on the screen of FIG. 5, the image of the small-section image 114 and the pathological-tissue image are arranged and displayed side by side on the pathological-tissue browsing screen on the upper side in the window W1. When the user clicks the small-section image 114, the pathological-tissue image display unit 410 changes the pathological-tissue browsing screen to a split screen display. In a display region 129 on the right side in the split screen, the small-section image 114 that is enlarged is displayed. In a display region 128 on the left side in the split screen, a part of the pathological-tissue image is displayed. At that time, the sub-region 109B related to the small-section image 114 is located at or near the center of the display region 128. This allows the user to more easily compare the small-section image of the past case with the image of the related sub-region.

(Example of Visualization of Sub-Region)

An example in which a sub-region set in an analysis target region is visualized to be easily distinguished from the analysis target region will be described.

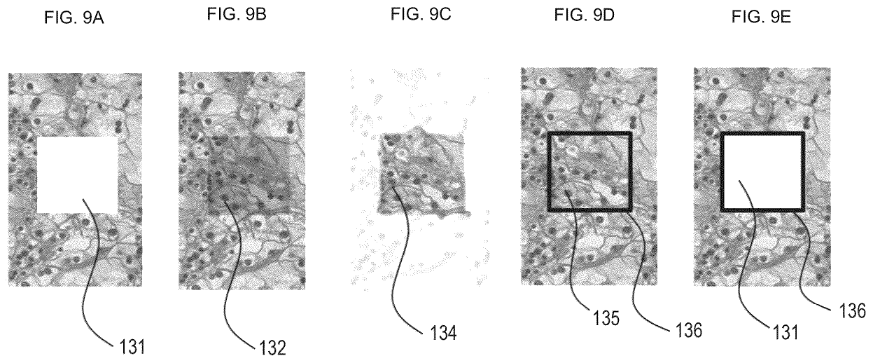

FIG. 9(A) illustrates an example in which a sub-region 131 is filled with a single color. This can enhance the visibility for the user in checking the sub-region. The color of the sub-region 131 is not limited to a specific color. Furthermore, the color may be changed for each sub-region by the user's operation. For example, the user can classify a sub-region using a randomly-selected reference (for example, the size of a cell included in the sub-region, or the like), and change the color of the sub-region according to each classification.

FIG. 9(B) illustrates an example in which the color transmittance of the sub-region 131 of FIG. 9(A) is changed. The sub-region in which the color transmittance has been changed is defined as a sub-region 132. To increase the transmittance allows the user to check the sub-region and check the structure of a pathological tissue in the sub-region at the same time. Furthermore, in a case where the color is changed according to a classification of a sub-region, it is possible to check the sub-region, check the structure of a pathological tissue in the sub-region, and check the classification of the sub-region at the same time.

FIG. 9(C) illustrates an example in which contrast between a sub-region 133 and its surrounding pathological-tissue image is increased. For example, by changing the hue, saturation, brightness, transparency, and the like of at least one of the sub-region 133 or the surrounding pathological-tissue image, it is possible to increase the contrast. This can enhance the visibility for the user without reducing the amount of information visually recognized in the sub-region 133. Furthermore, the user can refer to other information while observing the pathological tissue in the sub-region 133.

FIG. 9(D) illustrates an example in which the outer edge (boundary) of a sub-region 135 is enclosed with a single-color frame line 136. This can enhance the visibility of the sub-region. Furthermore, the display in the sub-region and its surrounding region is not changed, and hence it is easy to observe the sub-region and the surrounding pathological-tissue while comparing them.

FIG. 9(E) illustrates an example in which the examples illustrated in FIGS. 9(A) and 9(D) are combined. That is, the sub-region 131 is filled with a single color, and the outer edge of the sub-region 131 is enclosed with the single-color frame line 136. Furthermore, to selectively use a plurality of colors as the color of the sub-region 131 allows the user to perform color coding according to the classification of the sub-region, for example. In addition to the example illustrated in FIG. 9(E), various combinations are possible, which enables various expressions.

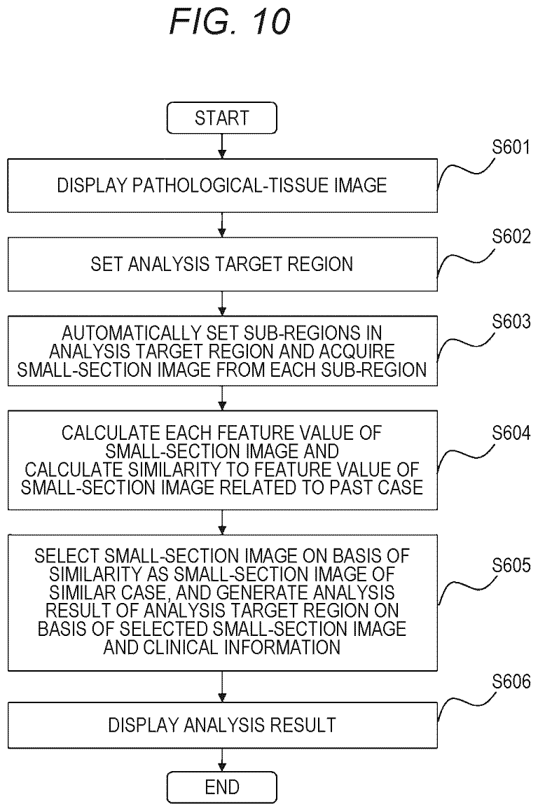

FIG. 10 is a flowchart schematically illustrating an example of overall operations of the medical image analysis apparatus 10 of the present disclosure.

The pathological-tissue image display unit 410 displays a pathological-tissue image selected by the user on the pathological-tissue browsing screen G1 (S601). The pathological-tissue image display unit 410 may further display clinical information and the like regarding the pathological-tissue image on the pathological-tissue browsing screen G1 or another screen.

The analysis-target-region setting unit 200 sets an analysis target region in the pathological-tissue image being displayed (S602).

The sub-region setting unit 300 sets one or more sub-regions in the analysis target region on the basis of the algorithm, and acquires an image of each sub-region (small-section image) (S603). Note that the acquired image may be enlarged or reduced for size normalization.

The similar-case retrieval unit 500 calculates a feature value from each small-section image, and calculates a similarity between the calculated feature value and the feature value of a small-section image related to a past case (S604). The similar-case retrieval unit 500 selects a small-section image on the basis of the calculated similarity, and sets the selected small-section image as a small-section image of a similar case. An analysis result of the analysis target region is generated on the basis of the selected small-section image and clinical information or the like (S605). The similar-case retrieval unit 500 provides the analysis result of the analysis target region to the case information display unit 420 (S606). The case information display unit 420 displays the analysis result of the analysis target region on the case information screen G2.

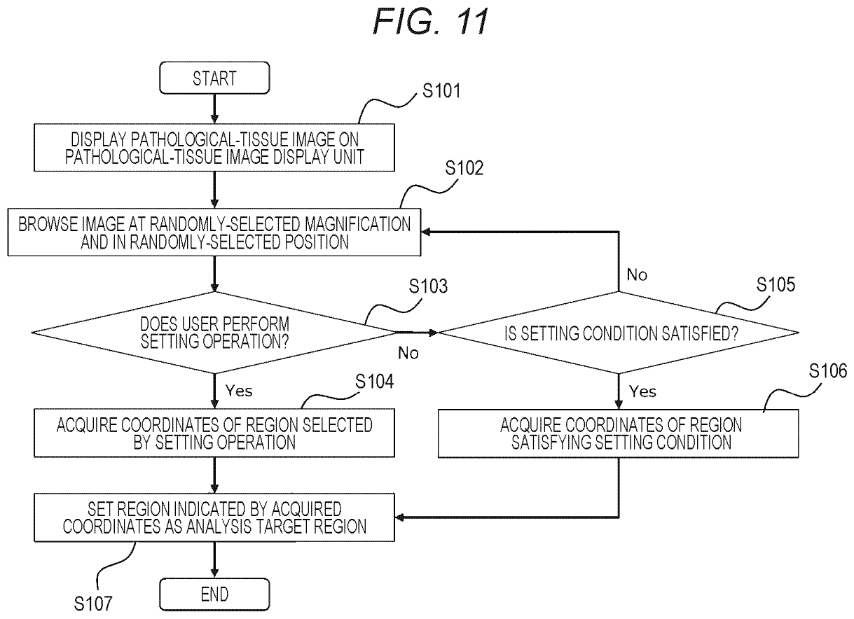

FIG. 11 is a flowchart illustrating an example of detailed operations of the pathological-tissue image display unit 410 and the analysis-target-region setting unit 200.

The pathological-tissue image display unit 410 reads a pathological-tissue image selected by the user from the diagnosis DB 40 and displays the image on the pathological-tissue browsing screen G1 in a window of the present application (S101) The user may display the pathological-tissue image at a randomly-selected magnification and in a randomly-selected position by operating the operation apparatus 20 (S102).

The analysis-target-region setting unit 200 determines whether or not the user has performed an operation of setting an analysis target region (S103). In a case where a setting operation has been performed, the process proceeds to step S104. In a case where a setting operation has not been performed, the process proceeds to step S105.

In step S104, the analysis-target-region setting unit 200 acquires coordinates of a region selected by the setting operation (S104), and the process proceeds to step S107.

In step S105, the analysis-target-region setting unit 200 determines whether or not the setting condition is satisfied. In a case where the setting condition is satisfied, the process proceeds to step S106. In a case where the setting condition is not satisfied, the process returns to step S102. For example, in a case where an image belonging to a predetermined region of the pathological-tissue browsing screen is kept displayed for a certain period of time or more, it is determined that the setting condition is satisfied. In step S106, the analysis-target-region setting unit 200 acquires the coordinates of the predetermined region in the image, and the process proceeds to step S107.

In step S107, the analysis-target-region setting unit 200 sets the region specified by the acquired coordinates as an analysis target region in the pathological-tissue image. The analysis-target-region setting unit 200 provides information about the set analysis target region to the sub-region setting unit 300.

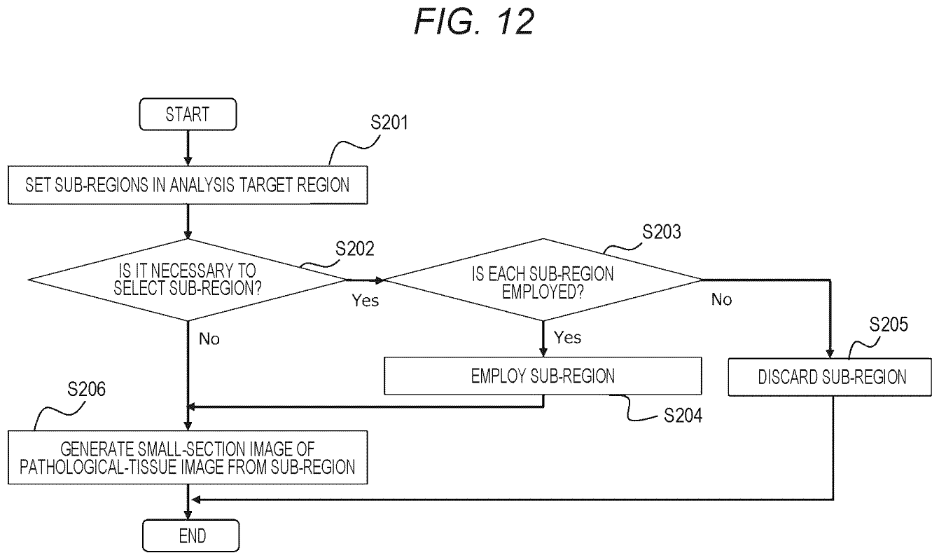

FIG. 12 is a flowchart illustrating an example of detailed operations of the sub-region setting unit 300.

The sub-region setting unit 300 sets one or more sub-regions (sample regions) in the analysis target region (S201). For example, coordinates are randomly selected from the analysis target region, and a sub-region is set using the selected coordinates as a reference. The sub-region setting unit 300 may additionally or alternatively determine a magnification different from a magnification of an image being browsed by the user, in accordance with a case desired to be retrieved, and set a sub-region in the image at the determined magnification.

The sub-region setting unit 300 determines whether or not it is necessary to perform selection on the sub-regions set in step S201 (S202). The selection of a sub-region is to select one or a plurality of representative sub-regions among the sub-regions set in step S201 and discard sub-regions other than the representative. Whether or not the selection of a sub-region is necessary may be determined on the basis of instruction information from the user, or may be determined autonomously by the sub-region setting unit 300. For example, in a case where the number of sub-regions is a certain value or more, it may be determined that the selection of a sub-region is necessary. By performing the selection, it is possible to reduce redundancy of the analysis result of the analysis target region and occurrence of duplication of retrieval results of sub-regions. Furthermore, the number of sub-regions to be processed is reduced, and hence there is produced an effect of reducing a processing load. In a case where the sub-region setting unit 300 determines that the selection of a sub-region is necessary, the sub-region setting unit 300 acquires an image (small-section image) of the sub-region from the pathological-tissue image on the basis of the set coordinates of the sub-region (S206).

On the other hand, in a case where it is determined that the selection of a sub-region is necessary, the sub-region setting unit 300 performs a selection process as to whether or not to employ each sub-region (S203). For example, a sub-region having a cell density of a certain value or more is employed, and the other sub-regions are discarded. Alternatively, only a sub-region including a cell nucleus having a size of a certain value or more is employed, and the other sub-regions are discarded. In addition, other methods described above are also possible. The sub-region setting unit 300 employs the sub-region that has been determined to be employed (S204). Then, the sub-region setting unit 300 acquires an image (small-section image) of the sub-region from the pathological-tissue image on the basis of the coordinates of the employed sub-region (S206). Meanwhile, the sub-region setting unit 300 discards the sub-region that has been determined not to be employed (S205).

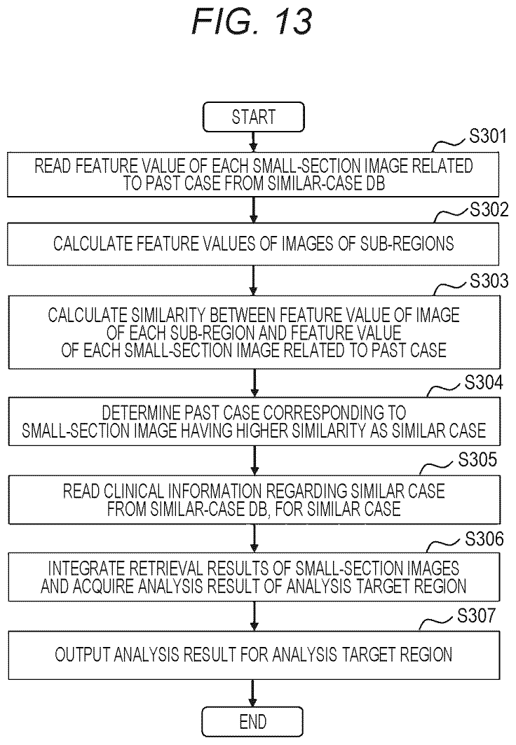

FIG. 13 is a flowchart illustrating an example of detailed operations of the similar-case retrieval unit 500.

The similar-case retrieval unit 500 reads the feature value of each small-section image related to a past case from the similar-case DB 30 (S301).

The similar-case retrieval unit 500 calculates the feature value of the image of each of the one or more sub-regions set by the sub-region setting unit 300 (S302).

The similar-case retrieval unit 500 calculates a similarity between the feature value of the image of each sub-region and the feature value of each small-section image related to the past case (S303).

The similar-case retrieval unit 500 determines a past case corresponding to a certain number of small-section images each having a higher similarity, as a similar case (S304).

The similar-case retrieval unit 500 reads clinical information regarding each similar case from the similar-case DB 30 (S305).

The similar-case retrieval unit 500 integrates the small-section image of each similar case and the clinical information to generate an analysis result for the analysis target region (S306). Note that, in a case where a similar case has been determined for one small-section image in step S304, it is only required to use the one small-section image and clinical information regarding the one small-section image, as an analysis result of the analysis target region.

The similar-case retrieval unit 500 outputs the analysis result for the analysis target region to the case information display unit 420 (S307). The case information display unit 420 displays the analysis result for the analysis target region on the case information screen G2 or the like in a window of the present application.

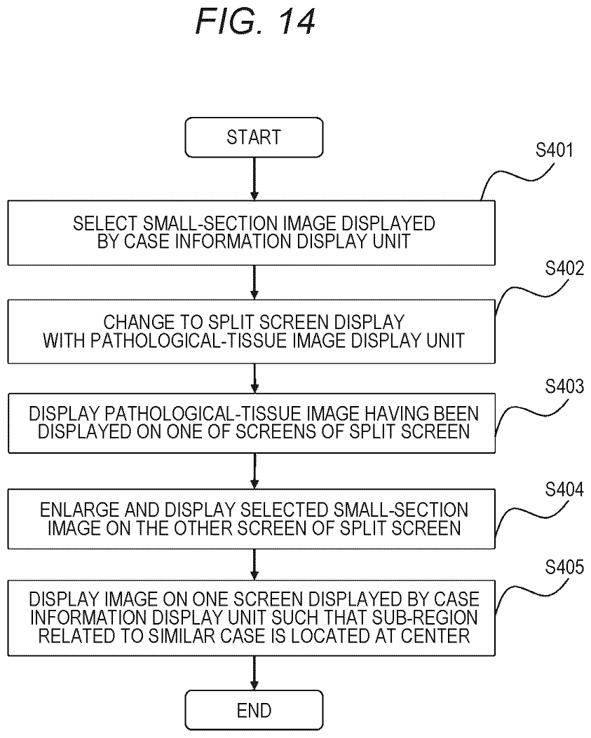

FIG. 14 is a flowchart illustrating an example of a display operation performed in a case where the user selects a small-section image of a similar case on the case information screen G2. Specifically, an example of operations performed for achieving the above-described display illustrated in FIG. 8 will be described.

The user selects (by a click or the like) a small-section image related to a similar case displayed on the case information screen G2. The output unit 400 acquires information for identifying the small-section image selected by the user (S401).

The pathological-tissue image display unit 410 changes the pathological-image display screen to a split screen display (S402).

The pathological-tissue image display unit 410 displays the pathological-tissue image displayed before the change on one of the screens (first display region) of the split screen (S403).

The pathological-tissue image display unit 410 enlarges and displays the small-section image selected in step S401 or the pathological-tissue image including a sub-region corresponding to the selected small-section image, or both of them, on the other of the screens (second display region) of the split screen (S404).

The pathological-tissue image display unit 410 adjusts the display position of the pathological-tissue image displayed in the first display region in the split screen such that the sub-region related to the selected small-section image (similar case) is located at the center (S405).

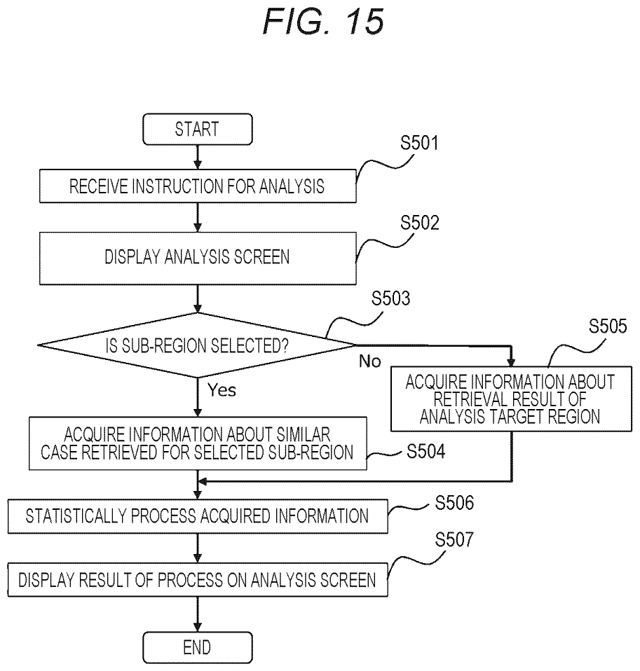

FIG. 15 is a flowchart illustrating an example of operations performed in which the similar-case retrieval unit 500 analyzes an image (an image of a sub-region or an entire image of an analysis target region) and displays an analysis result on the analysis screen G3 in the window.

The user clicks the analysis button displayed on the screen of the present application. The similar-case retrieval unit 500 detects an analysis instruction from the user on the basis of a click of the analysis button (S501). The medical image analysis apparatus 10 displays the analysis screen G3 in the screen of the present application or another screen to be separately activated (S502).

The case information display unit 420 determines whether or not any of sub-regions displayed on the pathological-tissue browsing screen is selected by the user (S503). In a case where any of the sub-regions is selected by the user, the process proceeds to step S504. In a case where none of the sub-regions is selected, the process proceeds to step S505.

In step S504, the output unit 400 acquires information (small-section image, clinical information, and the like) regarding the similar case retrieved with respect to the selected sub-region. Furthermore, in a case where a sub-region is not selected by the user, the output unit 400 acquires information regarding the analysis result of the analysis target region (S505).

The output unit 400 statistically processes the information acquired in step S504 or step S505 (S506). The details of the statistical process are as described above with reference to FIG. 7. The output unit 400 displays the statistical information including data generated by the statistical process on the analysis screen G3 (S507).

As described above, with the medical image analysis apparatus 10 of the present disclosure, a surgeon corresponding to the user, such as a pathologist, automatically retrieves and displays a similar case (an image of a similar case in the past and clinical information) from past cases using image information about a pathological-tissue image, while browsing the pathological-tissue image. This can improve the efficiency of surgeon's work in diagnosis and research.

(Modifications)

A part of the medical image analysis apparatus 10 may be placed as a server in a communication network such as a cloud or the Internet. For example, all or a part of the components in the medical image analysis apparatus 10 may be implemented by a server computer or a cloud connected via a communication network. In this case, a computer including the operation apparatus 20 and the display is placed on the user side and communicates to/from the above-described server via the communication network to transmit and receive data or information.

[Example of Application]

Below, an example of application of the above-described medical image analysis apparatus 10 will be described. Note that the above-described medical image analysis apparatus 10 can be applied also to a randomly-selected system, a randomly-selected apparatus, and a randomly-selected method other than a microscope system 600 described below.

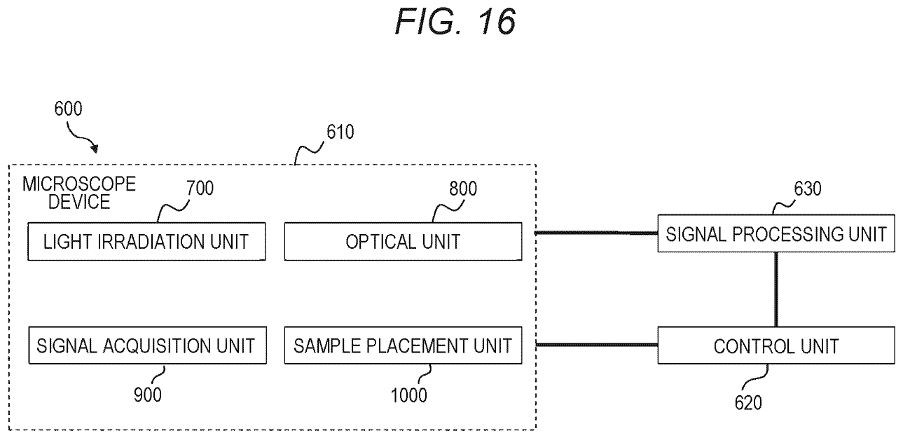

FIG. 16 is an example of a configuration of the microscope system 600 as an embodiment of the medical image analysis system of the present disclosure.

The microscope system 600 illustrated in FIG. 16 includes a microscope device 610, a control unit 620, and an information processing unit 630. In one example, the medical image analysis apparatus 10 or the medical image analysis system 100 of the present disclosure described above is implemented by the information processing unit 630 or both of the information processing unit 630 and the control unit 620. The microscope device 610 includes a light irradiation unit 700, an optical unit 800, and a signal acquisition unit 900. The microscope device 610 may further include a sample placement unit 1000 on which the biologically-originated sample S is placed. Note that the configuration of the microscope device 610 is not limited to that illustrated in FIG. 16. For example, the light irradiation unit 700 may be provided outside the microscope device 610, and for example, a light source not included in the microscope device 610 may be used as the light irradiation unit 700. Furthermore, the light irradiation unit 700 may be placed such that the sample placement unit 1000 is sandwiched between the light irradiation unit 700 and the optical unit 800, and for example, may be placed on a side where the optical unit 800 is provided. The microscope device 610 may include one or two or more of bright-field observation, phase difference observation, differential interference observation, polarization observation, fluorescence observation, and dark-field observation.

The microscope system 600 may be formed as a so-called whole slide imaging (WSI) system or a digital pathology system, and can be used for a pathological diagnosis. Furthermore, the microscope system 600 may be formed as a fluorescence imaging system, more specifically, a multiple fluorescence imaging system.

For example, the microscope system 600 may be used to perform an intraoperative pathological diagnosis or a remote pathological diagnosis. In the intraoperative pathological diagnosis, while surgery is being performed, the microscope device 610 can acquire data of the biologically-originated sample S acquired from a subject of the surgery and then transmit the data to the information processing unit 630. In the remote pathological diagnosis, the microscope device 610 can transmit the acquired data of the biologically-originated sample S to the information processing unit 630 located in a place away from the microscope device 610 (such as another room or building). Then, in these diagnoses, the information processing unit 630 receives and outputs the data. A user of the information processing unit 630 can perform a pathological diagnosis on the basis of the output data.

(Light Irradiation Unit)

The light irradiation unit 700 includes a light source for illuminating the biologically-originated sample S and an optical unit that guides light emitted from the light source to a specimen. The light source can irradiate a biologically-originated sample with visible light, ultraviolet light, infrared light, or a combination thereof. The light source may be one or two or more of a halogen lamp, a laser light source, an LED lamp, a mercury lamp, and a xenon lamp. A type and/or a wavelength of the light source in fluorescence observation may be plural, and may be appropriately selected by those skilled in the art. The light irradiation unit can have a transmissive, reflective, or epi-illumination (coaxial epi-illumination or trans-illumination) configuration.

(Optical Unit)

The optical unit 800 is configured to guide light from the biologically-originated sample S to the signal acquisition unit 900. The optical unit 800 can be configured to allow the microscope device 610 to observe or image the biologically-originated sample S.

The optical unit 800 can include an objective lens. A type of the objective lens may be appropriately selected by those skilled in the art according to an observation method. Furthermore, the optical unit 800 may include a relay lens for relaying an image enlarged by the objective lens to the signal acquisition unit 900. The optical unit 800 can further include an optical component other than the objective lens and the relay lens, an eyepiece lens, a phase plate, a condenser lens, and the like.

Furthermore, the optical unit 800 may further include a wavelength separation unit configured to separate light having a predetermined wavelength from light from the biologically-originated sample S. The wavelength separation unit can be configured to selectively cause light of a predetermined wavelength or a predetermined wavelength range to reach the signal acquisition unit. The wavelength separation unit may include, for example, one or two or more of a filter that selectively transmits light, a polarizing plate, a prism (Wollaston prism), and a diffraction grating. The optical component included in the wavelength separation unit may be placed, for example, on an optical path extending from the objective lens to the signal acquisition unit. The wavelength separation unit is provided in the microscope device in a case where fluorescence observation is performed, more specifically, in a case where an excitation-light irradiation unit is included. The wavelength separation unit can be configured to separate fluorescent light from each other or separate white light and fluorescent light.

(Signal Acquisition Unit)

The signal acquisition unit 900 can be configured to receive light from the biologically-originated sample S and convert the light into an electric signal, more specifically, a digital electric signal. The signal acquisition unit 900 may be configured to be capable of acquiring data regarding the biologically-originated sample S on the basis of the electric signal. The signal acquisition unit 900 may be configured to be capable of acquiring data of an image (an image, more specifically, a still image, a time-lapse image, or a moving image) of the biologically-originated sample S, and in particular, can be configured to acquire data of an image enlarged by the optical unit 800. The signal acquisition unit 900 includes an imaging device that includes one or a plurality of imaging elements, CMOS, CCD, or the like including a plurality of pixels arranged one-dimensionally or two-dimensionally. The signal acquisition unit 900 may include an imaging element for acquiring a low-resolution image and an imaging element for acquiring a high-resolution image, or may include an imaging element for sensing for AF or the like and an imaging element for image output for observation or the like. The imaging element can include a signal processing unit (including one, two, or three of a CPU, a DSP, and a memory) that performs signal processing using a pixel signal from each pixel, and an output control unit that controls output of image data generated from the pixel signal and processing data generated by the signal processing unit, in addition to the plurality of pixels. Moreover, the imaging element can include an asynchronous event detection sensor that detects, as an event, that a change of luminance of a pixel that photoelectrically converts incident light exceeds a predetermined threshold. The imaging element including the plurality of pixels, the signal processing unit, and the output control unit can be preferably formed as a one-chip semiconductor device.

(Control Unit)

The control unit 620 controls imaging by the microscope device 610. The control unit 620 can adjust a positional relationship between the optical unit 800 and the sample placement unit by driving movement of the optical unit 800 and/or the sample placement unit 1000 for imaging control. The control unit 620 can move the optical unit and/or the sample placement unit in a direction in which the optical unit and the sample placement unit get near to each other or go away from each other (for example, an optical-axis direction of the objective lens). Furthermore, the control unit may move the optical unit and/or the sample placement unit in any direction in a plane perpendicular to the optical-axis direction. The control unit may control the light irradiation unit 700 and/or the signal acquisition unit 900 for imaging control.

(Sample Placement Unit)

The sample placement unit 1000 may be configured to be capable of fixing a position of the biologically-originated sample S on the sample placement unit, and may be a so-called stage. The sample placement unit 1000 can be configured to be capable of moving the position of the biologically-originated sample S in the optical-axis direction of the objective lens and/or the direction perpendicular to the optical-axis direction.

(Information Processing Unit)

The information processing unit 630 can acquire data (such as imaging data) acquired by the microscope device 610, from the microscope device 610. The information processing unit 630 can perform image processing on imaging data. The image processing may include color separation processing. The color separation processing can include processing of extracting data of a light component of a predetermined wavelength or a predetermined wavelength range from imaging data to generate image data, processing of removing data of a light component of a predetermined wavelength or a predetermined wavelength range from imaging data, or the like. Furthermore, the image processing can include autofluorescence separation processing of separating an autofluorescence component and a pigment component of a tissue section, and fluorescence separation processing of separating wavelengths of pigments having different fluorescence wavelengths from each other. In the autofluorescence separation processing, processing may be performed in which an autofluorescence signal extracted from one of a plurality of specimens having the same or similar properties is used to remove an autofluorescence component from image information about another specimen.

The information processing unit 630 may transmit data for imaging control to the control unit 620, and the control unit 620 that has received the data may control imaging by the microscope device 610 in accordance with the data.

The information processing unit 630 may be formed as an information processing device such as a general-purpose computer, and may include a CPU, a RAM, and a ROM. The information processing unit may be included in a housing of the microscope device 610 or may be provided outside the housing. Furthermore, various kinds of processing or functions performed by the information processing unit may be implemented by a server computer or a cloud connected via a network.

A method of imaging the biologically-originated sample S by the microscope device 610 may be appropriately selected by those skilled in the art according to a type of the biologically-originated sample S, a purpose of imaging, or the like. An example of an imaging method will be described below.

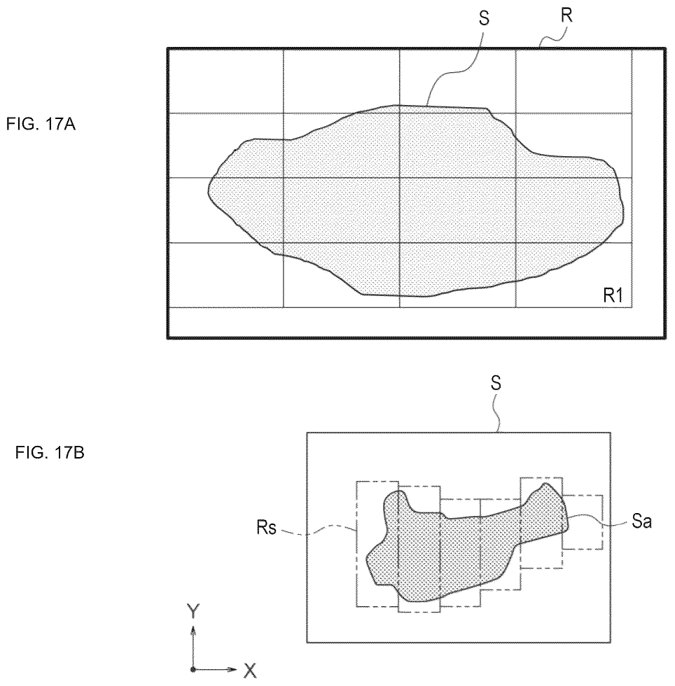

FIG. 17 is a view illustrating an example of an imaging method.

One example of an imaging method is as follows. The microscope device 610 can first specify an imaging target region. As the imaging target region, a region that covers an entire region where the biologically-originated sample S is present may be specified. Alternatively, a region that covers a target portion (a portion where a target tissue section, a target cell, or a target lesion is present) of the biologically-originated sample S may be specified. Subsequently, the microscope device 610 divides the imaging target region into a plurality of divisional regions each of a predetermined size, and the microscope device 610 sequentially images the divisional regions. Thus, an image of each divisional region is acquired.

As illustrated in FIG. 17(A), the microscope device 610 specifies an imaging target region R covering the entire biologically-originated sample S. Then, the microscope device 610 divides the imaging target region R into 16 divisional regions. Then, the microscope device 610 images a divisional region R1, and subsequently, can image any region in the regions included in the imaging target region R, such as a region adjacent to the divisional region R1. Then, the divisional regions are imaged until there is no divisional region left un-imaged. Note that a region other than the imaging target region R may also be imaged on the basis of captured image information about the divisional regions.

In order to image a next divisional region after imaging a certain divisional region, a positional relationship between the microscope device 610 and the sample placement unit is adjusted. The adjustment may be performed by movement of the microscope device 610, movement of the sample placement unit 1000, or movement of both. In this example, an imaging device that images each divisional region may be a two-dimensional imaging element (an area sensor) or a one-dimensional imaging element (a line sensor). The signal acquisition unit 900 may image each divisional region via the optical unit. Furthermore, imaging of each divisional region may be continuously performed while the microscope device 610 and/or the sample placement unit 1000 are moved. Alternatively, during imaging of each divisional image, movement of the microscope device 610 and/or the sample placement unit 1000 may be stopped. The imaging target region may be divided such that the divisional regions partly overlap each other, or the imaging target region may be divided such that the divisional regions do not overlap. Each divisional region may be imaged a plurality of times under different imaging conditions such as a focal length and/or an exposure time.

Furthermore, the information processing unit 630 can combine a plurality of adjacent divisional regions to generate image data of a wider region. By performing the combining process over the entire imaging target region, it is possible to acquire an image of a wider region for the imaging target region. Furthermore, image data with lower resolution can be generated from the image of the divisional region or the image obtained by the combining process.

Another example of the imaging method is as follows. The microscope device 610 can first specify an imaging target region. As the imaging target region, a region that covers an entire region where the biologically-originated sample S is present may be specified. Alternatively, a region that covers a target portion (a portion where a target tissue section or a target cell is present) in the biologically-originated sample S may be specified. Subsequently, the microscope device 610 scans and images a partial region (also referred to as a “divisional scan region”) of the imaging target region in one direction (also referred to as a “scan direction”) in a plane perpendicular to the optical axis. When the scan of the divisional scan region is completed, next, a divisional scan region adjacent to the scan region is scanned. Such a scanning operation is repeated until the entire imaging target region is imaged.

As illustrated in FIG. 17(B), the microscope device 610 specifies a region where a tissue section is present (a gray portion) in the biologically-originated sample S, as an imaging target region Sa. Then, the microscope device 610 scans a divisional scan region Rs in the imaging target region Sa in a Y-axis direction. When the scan of the divisional scan region Rs is completed, the microscope device next scans a divisional scan region adjacent to the divisional scan region Rs in an X-axis direction. This operation is repeated until scanning of the entire imaging target region Sa is completed.

A positional relationship between the microscope device 610 and the sample placement unit 1000 is adjusted in order to scan each divisional scan region and image a next divisional scan region after imaging a certain divisional scan region. The adjustment may be performed by movement of the microscope device 610, movement of the sample placement unit, or movement of both. In this example, an imaging device that images each divisional scan region may be a one-dimensional imaging element (a line sensor) or a two-dimensional imaging element (an area sensor). The signal acquisition unit 900 may image each divisional region via a magnifying optical system. Furthermore, the imaging of each divisional scan region may be continuously performed while the microscope device 610 and/or the sample placement unit 1000 are moved. The imaging target region may be divided such that the divisional scan regions partly overlap, or the imaging target region may be divided such that the divisional scan regions do not overlap. Each divisional scan region may be imaged a plurality of times under different imaging conditions such as a focal length and/or an exposure time.

Furthermore, the information processing unit 630 can combine a plurality of adjacent divisional scan regions to generate image data of a wider region. By performing the combining process over the entire imaging target region, it is possible to acquire an image of a wider region for the imaging target region. Furthermore, image data with lower resolution can be generated from the image of the divisional scan region or the image obtained by the combining process.

Note that the above-described embodiment has described examples for embodying the present disclosure, and the present disclosure can be implemented in various other forms. For example, various modifications, replacements, omissions, or combinations thereof can be made without departing from the gist of the present disclosure. Forms in which such modifications, replacements, omissions, and the like have been made are also included in the scope of the present disclosure and are likewise included in the invention described in the claims and the equivalent scopes thereof.

Furthermore, the effects of the present disclosure described in the present specification are mere examples, and other effects may be provided.

Note that the present disclosure can have the following configurations.

### [Item 1]

A medical image analysis apparatus including:
a first setting unit configured to set sample regions in an analysis target region of an image obtained by imaging of a biologically-originated sample, on the basis of an algorithm;
a processing unit configured to select at least one reference image from a plurality of reference images associated with a plurality of cases, on the basis of images of the sample regions; and an output unit configured to output the selected reference image.

### [Item 2]

The medical image analysis apparatus according to the item 1, in which
the first setting unit sets the sample regions in random positions in the analysis target region.

### [Item 3]

The medical image analysis apparatus according to the item 1 or 2, in which
the first setting unit sets the sample regions at equal intervals in the analysis target region.

### [Item 4]

The medical image analysis apparatus according to any of the items 1 to 3, in which
the first setting unit selects a sample region from the sample regions on the basis of a density of cell nuclei included in the sample regions, and
the processing unit selects the reference image on the basis of an image of the selected sample region.

### [Item 5]

The medical image analysis apparatus according to any of the items 1 to 4, in which
the first setting unit selects a sample region from the sample regions on the basis of a size of a cell nucleus included in the sample regions, and
the processing unit selects the reference image on the basis of an image of the selected sample region.

### [Item 6]

The medical image analysis apparatus according to any of the items 1 to 5, in which
the first setting unit clusters a plurality of the sample regions to generate a plurality of clusters, and selects the sample region from the clusters, and
the processing unit selects the reference image on the basis of an image of the sample region selected from the clusters.

### [Item 7]

The medical image analysis apparatus according to any of the items 1 to 6, in which
the first setting unit determines one or more magnifications corresponding to a case to be analyzed among a plurality of magnifications of an image, and
the first setting unit sets the sample region in the analysis target region of the image at the determined magnification.

### [Item 8]

The medical image analysis apparatus according to any of the items 1 to 7, in which
the processing unit calculates a similarity between the images of the sample regions and the reference images, and selects the reference image on the basis of the similarity.

### [Item 9]

The medical image analysis apparatus according to the item 8, in which
the processing unit calculates feature values of the images of the sample regions, and calculates the similarity on the basis of the feature values and feature values of the reference images.

### [Item 10]

The medical image analysis apparatus according to any of the items 1 to 9, further including:
a display unit configured to display a part or all of the image obtained by imaging of the biologically-originated sample; and
a second setting unit configured to set the analysis target region in the image displayed on the display unit.

### [Item 11]

The medical image analysis apparatus according to the item 10, in which
the second setting unit sets a predetermined range of the image displayed on the display unit as the analysis target region.

### [Item 12]

The medical image analysis apparatus according to the item 10 or 11, in which
the second setting unit sets the analysis target region in the image on the basis of instruction information from an operator.

### [Item 13]

The medical image analysis apparatus according to any of the items 8 to 12, in which
the output unit displays a part or all of the image obtained by imaging of the biologically-originated sample on a first screen portion in a screen of an application, and displays the selected reference image on a second screen portion in the screen of the application.

### [Item 14]

The medical image analysis apparatus according to the item 13, in which
the output unit places the selected reference image in the second screen portion in an order according to the similarity.

### [Item 15]

The medical image analysis apparatus according to the item 14, in which
the output unit selects one sample region from the sample regions on the basis of instruction information from an operator, and places an image of the selected sample region and the reference images for which the similarity has been calculated, on the second screen portion in an order according to the similarity to the image of the selected sample region.

### [Item 16]

The medical image analysis apparatus according to the item 14 or 15, in which
the output unit selects one reference image from the reference images displayed on the second screen portion on the basis of instruction information from an operator, and
the output unit displays the selected reference image and an image including the sample region for which the similarity to the selected reference image has been calculated, side by side.

### [Item 17]

The medical image analysis apparatus according to the item 1, in which
the plurality of reference images are associated with clinical information about the plurality of cases, and
the output unit further outputs the clinical information regarding the selected reference image.

### [Item 18]

A medical image analysis system including:
an imaging device configured to image a biologically-originated sample;
a first setting unit configured to set sample regions in an analysis target region of an image acquired by the imaging device, on the basis of an algorithm;
a processing unit configured to select at least one reference image from a plurality of reference images associated with a plurality of cases, on the basis of images of the sample regions; and
an output unit configured to output the selected reference image.

### [Item 19]

The medical image analysis system according to the item 18, further including
a computer program executed by a computer to cause the computer to function as the first setting unit, the processing unit, and the output unit.

### [Item 20]

A medical image analysis method including:
setting sample regions in an analysis target region of an image obtained by imaging a biologically-originated sample, on the basis of an algorithm:
selecting at least one reference image from a plurality of reference images associated with a plurality of cases, on the basis of images of the sample regions; and
outputting the selected reference image.

### REFERENCE SIGNS LIST

10 Medical image analysis apparatus
20 Operation apparatus
30 Similar-case database
40 Diagnosis database
100 Medical image analysis system
106 Region
107 Analysis target region
107_1 Analysis target region
109A Sub-region
109B Sub-region
109C Sub-region
109D Sub-region
111 to 116, 121 to 126 Small-section image
128, 129 Display region
131 to 133, 135 Sub-region
136 Frame line
G1 Pathological-tissue browsing screen
G2 Case information screen
G3 Analysis screen (analysis window)
141 Circle graph
142 Bar graph
200 Analysis-target-region setting unit (second setting unit)
300 Sub-region setting unit (first setting unit)
400 Output unit
410 Pathological-tissue image display unit
420 Case information display unit
500 Similar-case retrieval unit
600 Microscope system (medical image analysis system)
610 Microscope device
620 Control unit
630 Information processing unit
700 Light irradiation unit
800 Optical unit
900 Signal acquisition unit
1000 Sample placement unit

1. A medical image analysis apparatus comprising:
a first setting unit configured to set sample regions in an analysis target region of an image obtained by imaging of a biologically-originated sample, on a basis of an algorithm;
a processing unit configured to select at least one reference image from a plurality of reference images associated with a plurality of cases, on a basis of images of the sample regions; and
an output unit configured to output the selected reference image.

2. The medical image analysis apparatus according to claim 1, wherein
the first setting unit sets the sample regions in random positions in the analysis target region.

3. The medical image analysis apparatus according to claim 1, wherein
the first setting unit sets the sample regions at equal intervals in the analysis target region.

4. The medical image analysis apparatus according to claim 1, wherein
the first setting unit selects a sample region from the sample regions on a basis of a density of cell nuclei included in the sample regions, and
the processing unit selects the reference image on a basis of an image of the selected sample region.

5. The medical image analysis apparatus according to claim 1, wherein
the first setting unit selects a sample region from the sample regions on a basis of a size of a cell nucleus included in the sample regions, and
the processing unit selects the reference image on a basis of an image of the selected sample region.

6. The medical image analysis apparatus according to claim 1, wherein
the first setting unit clusters a plurality of the sample regions to generate a plurality of clusters, and selects the sample region from the clusters, and
the processing unit selects the reference image on a basis of an image of the sample region selected from the clusters.

7. The medical image analysis apparatus according to claim 1, wherein
the first setting unit determines one or more magnifications corresponding to a case to be analyzed among a plurality of magnifications of an image, and
the first setting unit sets the sample region in the analysis target region of the image at the determined magnification.

8. The medical image analysis apparatus according to claim 1, wherein
the processing unit calculates a similarity between the images of the sample regions and the reference images, and selects the reference image on a basis of the similarity.

9. The medical image analysis apparatus according to claim 8, wherein
the processing unit calculates feature values of the images of the sample regions, and calculates the similarity on a basis of the feature values and feature values of the reference images.

10. The medical image analysis apparatus according to claim 1, further comprising:
a display unit configured to display a part or all of the image obtained by imaging of the biologically-originated sample; and
a second setting unit configured to set the analysis target region in the image displayed on the display unit.

11. The medical image analysis apparatus according to claim 10, wherein
a position of the image displayed on the display unit is movable by an operator, and
the second setting unit sets a region of the image included in a predetermined region in a display region of the image as the analysis target region in a case where the image is kept unmoved for a certain period of time.

12. The medical image analysis apparatus according to claim 10, wherein
the second setting unit sets the analysis target region in the image on a basis of instruction information from an operator.

13. The medical image analysis apparatus according to claim 8, wherein
the output unit displays a part or all of the image obtained by imaging of the biologically-originated sample on a first screen portion in a screen of an application, and displays the selected reference image on a second screen portion in the screen of the application.

14. The medical image analysis apparatus according to claim 13, wherein
the output unit places the selected reference image in the second screen portion in an order according to the similarity.

15. The medical image analysis apparatus according to claim 14, wherein
the output unit selects one sample region from the sample regions on a basis of instruction information from an operator, and places the reference images for which the similarity to the selected sample region has been calculated, on the second screen portion in an order according to the similarity to the image of the selected sample region.

16. The medical image analysis apparatus according to claim 14, wherein
the output unit selects one reference image from the reference images displayed on the second screen portion on a basis of instruction information from an operator, and
the output unit outputs a split display screen in which the selected reference image and an image including the sample region for which the similarity to the selected reference image has been calculated are arranged and displayed.

17. The medical image analysis apparatus according to claim 1, wherein
the plurality of reference images are associated with clinical information about the plurality of cases, and
the output unit further outputs the clinical information regarding the selected reference image.

18. A medical image analysis system comprising:
an imaging device configured to image a biologically-originated sample;
a first setting unit configured to set sample regions in an analysis target region of an image acquired by the imaging device, on a basis of an algorithm;
a processing unit configured to select at least one reference image from a plurality of reference images associated with a plurality of cases, on a basis of images of the sample regions; and
an output unit configured to output the selected reference image.

19. The medical image analysis system according to claim 18, further comprising
a computer program executed by a computer to cause the computer to function as the first setting unit, the processing unit, and the output unit.

20. A medical image analysis method comprising:
setting sample regions in an analysis target region of an image obtained by imaging a biologically-originated sample, on a basis of an algorithm:
selecting at least one reference image from a plurality of reference images associated with a plurality of cases, on a basis of images of the sample regions; and
outputting the selected reference image.
